(19) United States
(12) Patent Application Publication
(10) Pub. No.: US 2026/0155244 A1
(43) Pub. Date: Jun. 4, 2026

(54) ENDOSCOPIC EXAMINATION ASSISTANCE METHOD, ENDOSCOPIC EXAMINATION ASSISTANCE DEVICE, AND RECORDING MEDIUM

(71) Applicant: OLYMPUS MEDICAL SYSTEMS CORP., Tokyo (JP)

(72) Inventors: Taketo TSUKIOKA, Hachioji-shi (JP); Osamu NONAKA, Machida-shi (JP); Shunsuke OKUMURA, Machida-shi (JP); Kai TANAKA, Nishitokyo-shi (JP)

(73) Assignee: OLYMPUS MEDICAL SYSTEMS CORP., Tokyo (JP)

(21) Appl. No.: 19/457,434

(22) Filed: Jan. 23, 2026

Related U.S. Application Data
(63) Continuation of application No. PCT/JP2023/027964, filed on Jul. 31, 2023.

(51) Int. Cl. G16H 40/20; A61B 1/00; A61B 34/20; G06T 7/00; G06T 7/73; G06V 10/764

(52) U.S. Cl. CPC G16H 40/20; A61B 1/00045; A61B 1/00131; A61B 34/20; G06T 7/0016; G06T 7/74; G06T 2207/10068; G06T 2207/20081; G06T 2207/30028; G06T 2207/30096; G06V 10/764; G06V 2201/031

(57) ABSTRACT

An endoscopic examination assistance method includes calculating a remaining time until an examination of an internal organ using an endoscope ends based on a preset examination time for the examination and an elapsed time having elapsed after the examination has started, acquiring current position information indicating a current examination position in the internal organ, and generating and outputting an examination time table including a scheduled examination time for each of a plurality of sections in the internal organ using the remaining time and the current position information.

### BACKGROUND OF THE INVENTION

### Field of the Invention

The present invention relates to an endoscopic examination assistance method, an endoscopic examination assistance device, and a recording medium.

This application is a continuation application based on PCT International Patent Application No. PCT/JP 2023/027964, filed Jul. 31, 2023, the content of which is incorporated herein by reference.

### Description of Related Art

PCT International Publication No. WO2019/078273 discloses a system that detects a region of interest including a target object from a medical image and prevents overlooking of the target object. When it is determined that the target object has been overlooked, the system notifies a user that overlooking has occurred.

### SUMMARY OF THE INVENTION

According to a first aspect of the present invention, an endoscopic examination assistance method includes: calculating a remaining time until an examination of an internal organ using an endoscope ends based on a preset examination time for the examination and an elapsed time having elapsed after the examination has started; acquiring current position information indicating a current examination position in the internal organ; and generating and outputting an examination time table including a scheduled examination time for each of a plurality of sections in the internal organ using the remaining time and the current position information.

According to a second aspect of the present invention, in the first aspect, the endoscopic examination assistance method may further include: classifying each of the plurality of sections as an examination-completed section of which the examination has been completed or an examination-scheduled section of which the examination has not been completed using the current position information; and generating and outputting the examination time table including the scheduled examination time for each examination-scheduled section according to the remaining time.

According to a third aspect of the present invention, in the second aspect, the endoscopic examination assistance method may further include: determining whether overlooking has occurred in an examination of the examination-completed section; and re-classifying the examination-completed section in which it is determined that the overlooking has occurred as the examination-scheduled section at the time of generation and output of the examination time table.

According to a fourth aspect of the present invention, in the second aspect, is the endoscopic examination assistance method may further include: detecting a lesioned part in each of the plurality of sections when the endoscope is inserted into a lumen of the internal organ; storing information of the detected lesioned part in a storage medium; determining whether overlooking of the lesioned part has occurred in an examination of the examination-completed section using the information stored in the storage medium when the endoscope is pulled out from the lumen; and re-classifying the examination-completed section including the lesioned part in which it is determined that the overlooking has occurred as the examination-scheduled section at the time of generation and output of the examination time table.

According to a fifth aspect of the present invention, in the second aspect, the endoscopic examination assistance method may further include: referring to a section of interest which is the examination-completed section corresponding to the current position information in the examination time table; and outputting instruction information for instructing an examiner to re-examine the section of interest when a time having required for an examination in the section of interest is shorter than the scheduled examination time of the section of interest.

According to a sixth aspect of the present invention, in the fifth aspect, the endoscopic examination assistance method may further include: re-classifying the section of interest as the examination-scheduled section; and updating and outputting the examination time table based on a time required for the re-examination.

According to a seventh aspect of the present invention, in the second aspect, the endoscopic examination assistance method may further include: acquiring a reference time table including the scheduled examination time for each of the plurality of sections from a storage medium; executing a process of updating the scheduled examination time in the examination-scheduled section on the reference time table using the remaining time and the current position information; and outputting the updated reference time table as the examination time table.

According to an eighth aspect of the present invention, in the seventh aspect, the endoscopic examination assistance method may further include: determining states of images acquired by the endoscope in the plurality of sections based on an inference model acquired through machine learning using images acquired by the endoscope and annotations indicating that observation of the images is insufficient as training data; generating the reference time table according to the states determined based on the inference model; and recording the generated reference time table in the storage medium.

According to a ninth aspect of the present invention, in the seventh aspect, the endoscopic examination assistance method may further include: calculating a first sum of the scheduled examination times in the examination-scheduled sections out of the scheduled examination times included in the reference time table before update; calculating a second sum of the scheduled examination times in the examination-scheduled sections out of the scheduled examination times included in the reference time table after update; generating speed information indicating an examination speed according to a result of comparison between the first sum and the second sum; and outputting the examination time table and the speed information to a display.

According to a tenth aspect of the present invention, in the first aspect, the endoscopic examination assistance method may further include: calculating the scheduled examination time for each examination-scheduled section according to an adapter attached to a distal end of the endoscope; and generating and outputting the examination time table including the calculated scheduled examination time.

According to an eleventh aspect of the present invention, in the first aspect, the endoscopic examination assistance method may further include outputting the examination time table and the current position information to a display.

According to a twelfth aspect of the present invention, an endoscopic examination assistance device includes a processor configured to: calculate a remaining time until an examination of an internal organ using an endoscope ends based on a preset examination time for the examination and an elapsed time having elapsed after the examination has started; acquire current position information indicating a current examination position in the internal organ; and generate and output an examination time table including a scheduled examination time for each of a plurality of sections in the internal organ using the remaining time and the current position information.

According to a thirteenth aspect of the present invention, there is provided a non-transitory computer-readable recording medium storing a program causing a computer to execute: calculating a remaining time until an examination of an internal organ using an endoscope ends based on a preset examination time for the examination and an elapsed time having elapsed after the examination has started; acquiring current position information indicating a current examination position in the internal organ; and generating and outputting an examination time table including a scheduled examination time for each of a plurality of sections in the internal organ using the remaining time and the current position information.

According to a fourteenth aspect of the present invention, an endoscopic examination assistance method includes: calculating a remaining time until an examination of an internal organ using an endoscope ends based on a preset examination time for the examination and an elapsed time having elapsed after the examination has started; acquiring current position information indicating a current examination position in the internal organ; and generating and outputting an examination time table including a scheduled examination time for each of a plurality of sections in the internal organ for simultaneously displaying the remaining time and the current position information.

According to a fifteenth aspect of the present invention, in the fourteenth aspect, the endoscopic examination assistance method may further include displaying the remaining time and a time distribution for an examination in each part of the internal organ in at least a region in which the remaining time is displayed based on the examination time table.

According to a sixteenth aspect of the present invention, in the fifteenth aspect, the time distribution may be determined according to a difference between surface areas of examination target parts of the internal organ to be examined, statistics of lesion occurrence of the examination target parts, or examination difficulty based on a shape of each examination target part.

According to a seventeenth aspect of the present invention, in the fourteenth aspect, the endoscopic examination assistance method may further include resetting the time distribution for the examination in each part of the internal organ based on the remaining time when an examination delay exceeding a predetermined time width is detected in a time allocated to the examination of each part of the internal organ.

According to an eighteenth aspect of the present invention, in the fifteenth aspect, the endoscopic examination assistance method may further include: classifying each of the plurality of sections as an examination-completed section of which the examination has been completed or an examination-scheduled section of which the examination has not been completed using the current position information; detecting a lesioned part in each of the plurality of sections when the endoscope is inserted into the internal organ; storing information of the detected lesioned part in a storage medium; determining whether overlooking of the lesioned part has occurred in an examination of the examination-completed section using the information stored in the storage medium when the endoscope is extracted from the internal organ; re-classifying the examination-completed section including the lesioned part in which it is determined that the overlooking has occurred as the examination-scheduled section at the time of generation and output of the examination time table; and displaying the remaining time and the time distribution for the examination in the examination-scheduled section.

According to a nineteenth aspect of the present invention, in the fifteenth aspect, the endoscopic examination assistance method may further include: classifying each of the plurality of sections as an examination-completed section of which the examination has been completed or an examination-scheduled section of which the examination has not been completed using the current position information; detecting an image feature in each of the plurality of sections when the endoscope is inserted into the internal organ; storing information of the detected image feature in a storage medium; and switching the time distribution for the examination in each part of the internal organ using the information of the image feature for each section stored in the storage medium.

According to a twentieth aspect of the present invention, in the fifteenth aspect, the endoscopic examination assistance method may further include: referring to the examination time table; and switching the time distribution for the examination in each part of the internal organ when a time having required for the examination of a specific section out of the plurality of sections in the internal organ is short.

### BRIEF DESCRIPTION OF THE DRAWINGS

FIG. 1 is a block diagram showing an example of the configuration of an endoscope system according to a first embodiment of the present invention.

FIG. 2 is a flowchart showing an example of a procedure of a process executed by an endoscopic examination assistance device according to the first embodiment of the present invention.

FIG. 3 is a diagram showing an example of a reference time table in the first embodiment of the present invention.

FIG. 4 is a diagram showing an example of a screen of a display unit in the first embodiment of the present invention.

FIG. 5 is a diagram showing a method of generating an inference model in the first embodiment of the present invention.

FIG. 6A is a diagram showing a state of an endoscope to which an adapter is attached in a second embodiment of the present invention.

FIG. 6B is a diagram showing a state of an endoscope to which an adapter is attached in the second embodiment of the present invention.

FIG. 7 is a flowchart showing an example of a procedure of a process executed by an endoscopic examination assistance device according to the second embodiment of the present invention.

FIG. 8 is a flowchart showing an example of a procedure of a process executed by the endoscopic examination assistance device according to the second embodiment of the present invention.

FIG. 9 is a diagram showing an example of a screen of a display unit in the second embodiment of the present invention.

FIG. 10A is a diagram showing a state of an endoscope to which an adapter is attached in the second embodiment of the present invention.

FIG. 10B is a diagram showing a state of an endoscope to which an adapter is attached in the second embodiment of the present invention.

FIG. 11 is a diagram showing an example of the screen of the display unit in the second embodiment of the present invention.

FIG. 12 is a diagram showing an example in which a lesioned part is treated in the second embodiment of the present invention.

FIG. 13 is a diagram showing an example of the screen of the display unit in the second embodiment of the present invention.

### DETAILED DESCRIPTION OF THE INVENTION

Hereinafter, embodiments of the present invention will be described with reference to the drawings. In the following description, an example of an endoscope system including an endoscopic examination assistance device will be described. “A plurality of XX” in the following description means two or more XX.

### First Embodiment

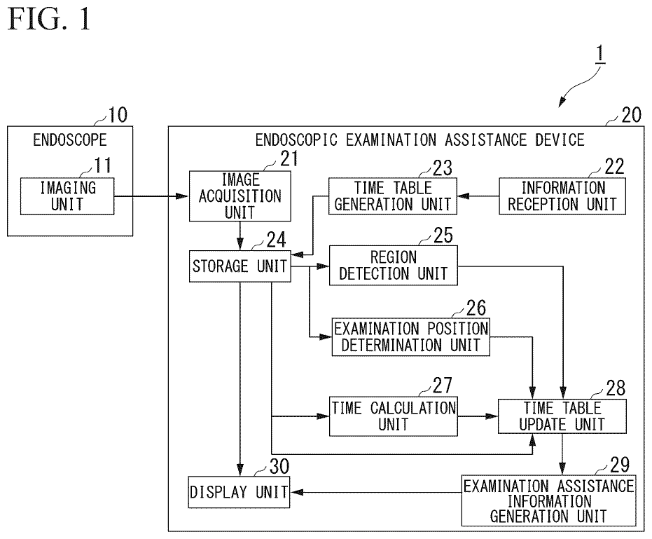

FIG. 1 shows an example of the configuration of an endoscope system 1 according to a first embodiment of the present invention. The endoscope system 1 shown in FIG. 1 includes an endoscope 10 and an endoscopic examination assistance device 20.

The endoscope 10 is inserted into an internal organ of an examinee. The internal organ is often a lumen. For example, the internal organ is an intestine such as the large intestine or the stomach. The endoscope 10 includes an imaging unit 11. The imaging unit 11 is disposed at the distal end of the endoscope 10. The imaging unit 11 is an image sensor and generates a plurality of images at a plurality of positions in the intestine of the examinee. The imaging unit 11 outputs the plurality of images to the endoscopic examination assistance device 20. An examiner (a doctor) inserts the endoscope 10 into the intestine of the examinee and causes the distal end of the endoscope 10 (which may be abbreviated to a distal end in the following description) to advance to a predetermined position. Thereafter, the examiner performs an examination while slowly drawing the endoscope 10.

The endoscopic examination assistance device 20 includes an image acquisition unit 21, an information reception unit 22, a time table generation unit 23, a storage unit 24, a region detection unit 25, an examination position determination unit 26, a time calculation unit 27, a time table update unit 28, an examination assistance information generation unit 29, and a display unit 30.

The image acquisition unit 21 sequentially acquires a plurality of images output from the imaging unit 11. The image acquisition unit 21 outputs the plurality of acquired images to the storage unit 24.

The information reception unit 22 receives information designated by an examiner. For example, the information reception unit 22 receives examination condition information indicating examination conditions and outputs the examination condition information to the time table generation unit 23. For example, the examination condition information includes sex of an examinee, age of the examinee, an examination target, and anesthesia information. The examination target indicates the type (a large intestine or the stomach) of an intestine. The anesthesia information indicates whether anesthesia is used in the examination.

The time table generation unit 23 generates a reference time table based on the examination condition information. The reference time table includes a plurality of divided sections into which an internal organ is positionally divided and includes a scheduled examination time required for an examination of each divided section. Each divided section indicates a part through which the distal end of the endoscope 10 passes in the examination. When the examination target is a large intestine, the part is an appendix, an ascending colon, a transverse colon, or a descending colon, or the like. The time table generation unit 23 outputs the generated reference time table to the storage unit 24. Details of the reference time table will be described later.

The storage unit 24 is a memory. The storage unit 24 stores the plurality of images output from the image acquisition unit 21 and the reference time table output from the time table generation unit 23. When the reference time table is updated by the time table update unit 28, the storage unit 24 stores the updated reference time table. The plurality of images stored in the storage unit 24 are sequentially output to the region detection unit 25, the examination position determination unit 26, and the display unit 30. The reference time table stored in the storage unit 24 is output to the time calculation unit 27 and the time table update unit 28.

The region detection unit 25 detects a feature region in each of a plurality of parts in the intestine based on the plurality of images output from the storage unit 24. In the following description, it is assumed that the region detection unit 25 detects an overlooked region as the feature region. A region in the intestine may not appear in an image or visibility of a region in the image may be poor. Alternatively, a region in the intestine appears in an image, but an examiner may not perform detailed observation or treatment of the region. The region detection unit 25 detects such a region as an overlooked region. When an overlooked region is detected, the region detection unit 25 outputs overlook information indicating occurrence of the overlooked region to the time table update unit 28.

The examination position determination unit 26 determines a position that is currently being examined based on the plurality of images output from the storage unit 24 and generates current position information indicating the position. The position indicates a position in the intestine that is being imaged by the imaging unit 11.

The “current examination position” may be a unit of an organ name (a part name) or a subdivided position in the internal organ. For example, the organ name is throat, stomach, or the like in the case of an upper intestine, and the organ name is rectum, transverse colon, or the like in the case of a lower intestine. For example, the subdivided position is a position advanced by about 3 cm in the transverse colon after passing through an S-shaped colon. The “currently examined position” may be a position in the lumen, a name in the lumen, or a classified position in the lumen. The “currently examined position” may be a combination of a name in the lumen and a position, a depth, a length, a distance, or the like in a lumen direction in the lumen. A direction perpendicular to a continuation direction (an axial direction) of the lumen may be used according to necessity.

In a bag-shaped organ such as the stomach, the “current examination position” may be a region name or a part name such as the cardia, the gastric fundus, the gastric corpus, the pyloric antrum, or the pylorus from an entrance or may be information indicating an arbitrary position of a corresponding part by coordinates or the like in more detail. This position information may be detected from image features of tissues, blood vessels, or the like that are specific to the corresponding part. The position may be determined using the change of image features accompanying the change of image frames consecutively acquired at the time of insertion of the endoscope 10 or using a pattern of blood vessels or tissues. The endoscopic examination assistance device 20 may detect a speed (cm/sec) or the like at the time of insertion of the endoscope 10 and convert the insertion position using time information, but may not detect the speed and use an average value of an insertion speed or the like as a constant when a general doctor inserts the endoscope. The endoscopic examination assistance device 20 may determine a lumen shape using three-dimensional (3D) reconstruction using image information or the like and estimate the current examination position based on the result of determination.

In the following example, the position indicates a specific part in a simplified intestine. When the endoscope 10 includes a position sensor at the distal end thereof, the examination position determination unit 26 may determine a part that is currently being examined based on information output from the position sensor. For example, there is a method of detecting a part using magnetism or the like. The part may be detected using another medical instrument such as a CT or an MRI. The examination position determination unit 26 generates part information (current position information) indicating the determined part and outputs the part information to the time table update unit 28.

An expression “a position in an internal organ,” may represent a region name in the internal organ such as the cardia, the gastric fundus, the gastric corpus, the pyloric antrum, or the pylorus. The “position in an internal organ” may be used to mean the difference between internal organs in different classifications such as the throat and the stomach.

The time calculation unit 27 calculates a remaining time based on a preset examination time and a time (an elapsed time) having elapsed after the examination has started. The remaining time is a time required for an examination until the examination ends. The examination time is a scheduled time required for the whole examination. The time calculation unit 27 calculates the remaining time by subtracting the elapsed time from the examination time. The time calculation unit 27 outputs the remaining time to the time table update unit 28.

The time table update unit 28 acquires the reference time table from the storage unit 24. When the overlook information is output from the region detection unit 25, the time table update unit 28 updates the reference time table based on both the part information output from the examination position determination unit 26 and the remaining time output from the time calculation unit 27.

When an overlooked region occurs in a specific part, it is assumed that the examiner returns the distal end of the endoscope 10 to a position at which the overlooked region can be observed from the current examination position (a current position in the internal organ, that is, a part of the internal organ in which the distal end of the endoscope 10 is currently located) and perform an examination at the position again. Since the examination is performed again, a time required for the examination at the part increases. In order to end the examination before an effect of anesthesia is cut, the whole examination time has to be within a predetermined time. However, when a time required for an examination in a specific part increases, there is a likelihood that the whole examination time may not be within the predetermined time.

This reconfirmation or necessary countermeasures are performed. The countermeasures are, for example, removal of residues, removal of bubbles, irrigation, water supply, removal of polyps, or hemostasis. Since an examinee appeals for something in the course of them or change of a biological signal is detected, the examination may be delayed from the scheduled time. However, since the remaining parts requiring an examination has to be reliably examined even when a delay occurs, it is important to be aware of management of a time table. In the present invention, an examiner can perform an examination with this awareness. The time table update unit 28 adjusts a scheduled examination time in a part of which the examination has not ended such that the whole examination time is within a predetermined time. In this way, the time table update unit 28 executes a process of updating the scheduled examination time in a part of which the examination has not ended on the reference time table using the remaining time and the part information.

When the overlook information is output from the region detection unit 25, the time table update unit 28 outputs the updated reference time table (an actual examination time table) to the storage unit 24 and the examination assistance information generation unit 29. When the overlook information is not output from the region detection unit 25, the time table update unit 28 outputs the non-updated reference time table to the examination assistance information generation unit 29. The time table update unit 28 outputs the overlook information output from the region detection unit 25 and the part information output from the examination position determination unit 26 to the examination assistance information generation unit 29.

The examination assistance information generation unit 29 generates examination assistance information including the reference time table and the part information output from the time table update unit 28. The examination assistance information generation unit 29 determines an examination state in each part. When the distal end of the endoscope 10 has passed through a specific part but it is determined that an examination in the part has not been completed, the examination assistance information generation unit 29 generates examination assistance information including instruction information for instructing an examiner to re-examine the part. For example, when the overlook information is output from the time table update unit 28, the examination assistance information generation unit 29 generates examination assistance information including a message for encouraging the examiner to re-examine a part in which an overlooked region has occurred. The examination assistance information generation unit 29 outputs the examination assistance information to the display unit 30.

The display unit 30 is a liquid crystal monitor or the like. The display unit 30 sequentially displays a plurality of images output from the storage unit 24. The display unit 30 displays the examination assistance information output from the examination assistance information generation unit 29 along with the images.

At least one of the image acquisition unit 21, the information reception unit 22, the time table generation unit 23, the region detection unit 25, the examination position determination unit 26, the time calculation unit 27, the time table update unit 28, and the examination assistance information generation unit 29 may be constituted by a processor such as a central processing unit (CPU).

A computer may read a program and execute the read program. The program includes instructions for defining operations of at least one of the image acquisition unit 21, the information reception unit 22, the time table generation unit 23, the region detection unit 25, the examination position determination unit 26, the time calculation unit 27, the time table update unit 28, and the examination assistance information generation unit 29. That is, the function of at least one of the image acquisition unit 21, the information reception unit 22, the time table generation unit 23, the region detection unit 25, the examination position determination unit 26, the time calculation unit 27, the time table update unit 28, and the examination assistance information generation unit 29 may be realized by software.

The program may be provided, for example, using a “computer-readable recording medium” such as a flash memory. The program may be transmitted from a computer storing the program to the endoscope system 1 via a transmission medium or using carrier waves in the transmission medium. The “transmission medium” for transmitting a program is a medium having a function of transmitting information. The medium having a function of transmitting information includes a network (a communication network) such as the Internet and a communication circuit line (a communication line) such as a telephone line. The program may realize some of the above-described functions. The program may be a differential file (a differential program). The above-described functions may be realized by combining the differential program with a program stored in advance in the computer.

The processor and the recording medium do not have to be included in one device and may be constituted by linking a plurality of devices having distributed functions. The processor and the recording medium may be provided on cloud (network).

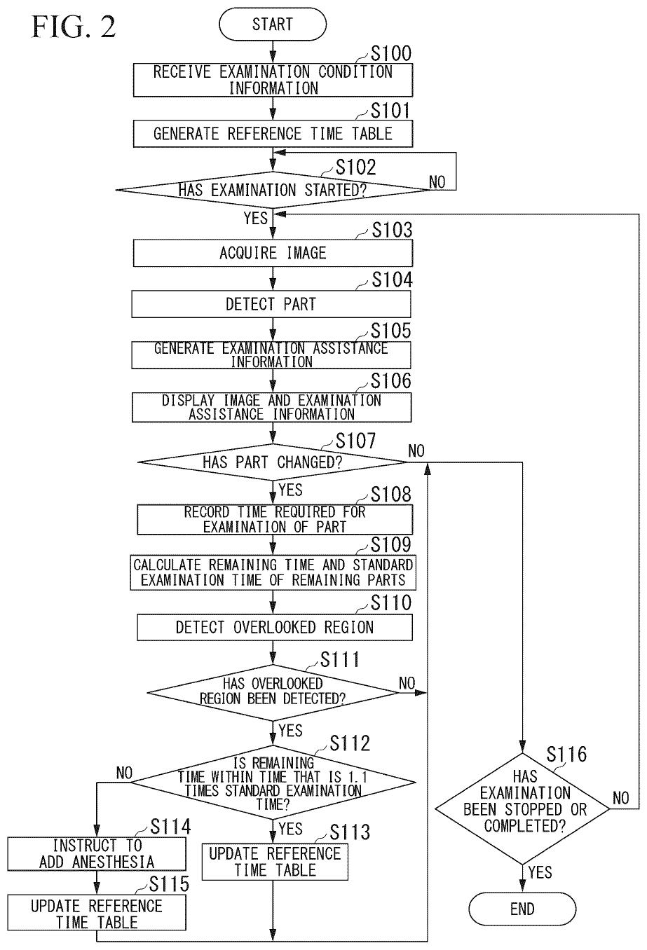

An example of operations of the endoscopic examination assistance device 20 will be described below with reference to FIG. 2. FIG. 2 shows an example of a procedure of a process executed by the endoscopic examination assistance device 20.

### Step S100

The information reception unit 22 receives examination condition information indicating examination conditions and outputs the examination condition information to the time table generation unit 23. As described above, the examination condition information includes sex of an examinee, age of an examinee, an examination target, and anesthesia information.

### Step S101

The time table generation unit 23 generates a reference time table based on the examination condition information and outputs the generated reference time table to the storage unit 24.

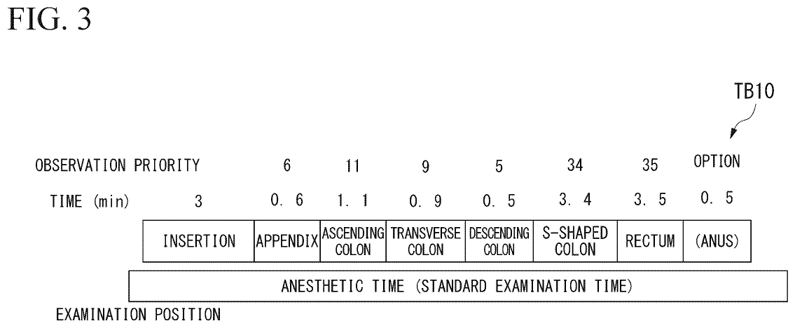

FIG. 3 shows an example of a reference time table. In the example shown in FIG. 3, the examination target is a large intestine. An examiner inserts the endoscope 10 into the large intestine and causes the distal end of the endoscope 10 to advance to the appendix. After the distal end of the endoscope 10 has reached the appendix, the examiner performs an examination while slowly drawing the endoscope 10. A reference time table TB10 shown in FIG. 3 includes a scheduled time required for insertion of the endoscope 10 and a scheduled time required for an examination of each part of the large intestine.

When anesthesia is used, the examination needs to be ended before an effect of the anesthesia is cut. Accordingly, a standard examination time based on a time in which the effect of anesthesia is maintained (a standard examination time from start to end of an examination) is preset. The anesthesia is not essential. Since use of anesthesia may be prohibited based on a patient's desire or constitution or a doctor's decision or the amount of anesthesia may be increased or decreased, an “anesthesia time” in the drawing is an indication of a reference. The standard scheduled time required for insertion of the endoscope 10 (an insertion time) is preset. The time table generation unit 23 determines a scheduled time for each part such that the sum of the scheduled times for the parts is less than or equal to the standard examination time other than the insertion time. At this time, the time table generation unit 23 determines the scheduled time for each part according to the priority of each part of the large intestine. The priority of each part is preset according to occurrence probabilities of a lesioned part in the part, the length of each part, and the like. The priorities (observation priorities) for the parts and the scheduled times of the parts are shown in FIG. 3. The time table generation unit 23 generates the reference time table including the insertion time and the scheduled times of the parts.

The display unit 30 has a feature of displaying the remaining time and a time distribution of an examination in each part of the internal organ in at least a region in which the remaining time is displayed based on the reference time table when the examination assistance information including the reference time table is output to the display unit 30. The time distribution represents times allocated in advance to an examination of each part of the internal organ. The reference time table is referred to for managing times required for an examination of each part to be examined from now on when a doctor or a medical employee performs an endoscopic examination. The time distribution other than the remaining time represents the times allocated in advance to an examination of the parts of which the examination has been completed. That time distribution is not referred to in the future and thus does not need to be displayed, but may be used for a report or the like along with an actual examination time.

The endoscopic examination assistance method, the endoscopic examination assistance device, and the program realize a process of calculating a remaining time until an examination ends based on both a preset examination time in an examination of an internal organ using an endoscope and an elapsed time having elapsed after the examination has started, acquiring current position information indicating a current examination position in a corresponding internal organ determined in an examination menu, and generating and outputting an examination time table for simultaneously displaying the remaining time and the current position information. The preset examination time may be adjustable according to an examinee's profile (such as stature, weight, sex, race, age, or constitution), a duration time of an anesthesia effect, and the like. The examination menu may be determined according to a medical checkup or an examination of a specific disease, or a doctor may determine the examination menu according to situations.

Managing examination times enables an efficient examination with no leakage in an appropriate time distribution and no schedule change. Even when too much time has been required for an examination of a specific part, the examiner can check a schedule of a time distribution thereafter and also easily recover the examination. A reference for such a time distribution is presented according to a doctor's experience or the like. Since the current position is changed and displayed from time to time with movement of the distal end of the endoscope 10, the examiner can easily predict the pace of the examination. Accordingly, even when treatment or the like is necessary and a time is required in the course of the examination, the examiner can see a guide of the time distribution and determine how fast subsequent examinations are to be performed.

The endoscopic examination assistance device 20 determines the time distribution according to the difference between surface areas of examination target parts in the internal organ to be examined, statistics of lesion occurrence of the examination target parts, or examination difficulty depending on shapes of the examination target parts. The preset examination time in an examination of the internal organ is determined based on a similar idea. When the examination proceeds smoothly in the scheduled times without any particular overlook, the original time distribution may be maintained. When exceeding of an estimated time exceeding the original time distribution is detected, that is, when an examination delay of a time greater than a predetermined time width with respect to the time allocated to an examination of each part of the internal organ is detected, the endoscopic examination assistance device 20 resets the time distribution of an examination in each part of the internal organ based on the remaining time until the examination ends.

In an endoscopic examination of an intestine, the examination of an internal organ is often an examination in a lumen of the internal organ, and the divided parts are often parts divided in a longitudinal direction of the lumen. A bag-shaped internal organ such as the stomach may be considered as a lumen of a complicated shape. This reference time table is helpful in determining whether an examination of each part has been performed in an appropriate time. Accordingly, the endoscopic examination assistance device 20 may refer to the reference time table, determine that the estimated time distribution has not been satisfied, and switch the time distribution of an examination in each part of the internal organ when a time having required for an examination of a specific part is short. This is because a part of which the examination time is short needs to be examined again or the whole examination needs to be reviewed but the examination time is limited. The reference time table serves as a reference of an examination time table, but the reference may be changed according to situations, which may be referred to as an examination time table.

Each part in the reference time table and the actual examination time table is classified as an examination-completed part (an examination-completed section) that is a part (divided section) of which an examination has been completed or an examination-scheduled part (an examination-scheduled section) that is a part (divided section) of which an examination has not been completed. The examination-scheduled part represents a part of which an examination has not started or a part of which an examination is being performed and has not been completed. All parts in the reference time table are examination-scheduled parts. The reference time table and the actual examination time table include part type information indicating the examination-completed part or the examination-scheduled part.

There is a likelihood that the length of each part or a lesion occurrence probability in each part will differ according to sex and age of an examinee. The time table generation unit 23 may change the scheduled time of each part in the reference time table according to sex and age included in the examination condition information.

The examination condition information may include past examination results of examinees. There is a likelihood that a lesioned part will be detected again in a part in which a lesioned part was detected in the past. Since there is a time required for an examination of that part will extend, the time table generation unit 23 may change the scheduled time of each part in the reference time table according to the past examination results.

### Step S102

When the examinee is anesthetized and the examination thereof starts, Step S103 described later is executed. A timepiece in the endoscopic examination assistance device 20 starts time measurement and generates time information indicating the measured time. When the examination has not started, the endoscopic examination assistance device 20 waits until the examination starts.

When the examination has started, the examiner may notify the endoscopic examination assistance device 20 that the examination has started by operating a button or the like. When the examiner has notified that the examination has started, the endoscopic examination assistance device 20 may execute Step S103.

### Step S103

The image acquisition unit 21 acquires an image output from the imaging unit 11. The image acquisition unit 21 outputs the image to the storage unit 24. The image output from the image acquisition unit 21 is output to the storage unit 24 and then is output to the region detection unit 25, the examination position determination unit 26, and the display unit 30.

### Step S104

The examination position determination unit 26 determines a current examination part based on the image output from the storage unit 24 and generates part information indicating the part. When the distal end or the like of the endoscope 10 includes a position sensor, the examination position determination unit 26 may determine the current examination part based on information output from the position sensor. The examination position determination unit 26 outputs the part information to the time table update unit 28.

### Step S105

The time table update unit 28 acquires the reference time table from the storage unit 24 and outputs the reference time table to the examination assistance information generation unit 29. When Step S113 or S115 described later has been executed, the time table update unit 28 acquires the reference time table updated in Step S113 or S115 and stored in the storage unit 24 from the storage unit 24 and outputs the reference time table to the examination assistance information generation unit 29. The time table update unit 28 outputs the part information output from the examination position determination unit 26 to the examination assistance information generation unit 29. The examination assistance information generation unit 29 generates examination assistance information including the reference time table and the part information output from the time table update unit 28 and outputs the examination assistance information to the display unit 30.

### Step S106

The display unit 30 displays the image output from the storage unit 24 and the examination assistance information output from the examination assistance information generation unit 29. An example of the examination assistance information displayed on the display unit 30 will be described later.

### Step S107

When the part determined by the examination position determination unit 26 has changed, Step S108 described later is executed. That is, when the current examination part has changed, Step S108 is executed. When the current examination part has not changed, Step S116 described later is executed. For example, while the appendix is being examined, the current examination part does not change. When the distal end of the endoscope 10 leaves the appendix and enters the ascending colon, the current examination part changes. That is, an examination of the appendix ends, and an examination of the ascending colon starts. At this time, Step S108 is executed.

### Step S108

The time calculation unit 27 acquires the time information from the timepiece in the endoscopic examination assistance device 20 and calculates a time (an elapsed time) having elapsed after the examination has started. The time calculation unit 27 calculates a time having required for an examination of a part of which an examination has been completed. Immediately after an examination of a first part has ended, a time having required for an examination of the part is the same as the elapsed time. Immediately after an examination of an N-th part has ended, a time having required for an examination of the N-th part is the same as a time obtained by subtracting the sum of times having required for an examination of the first to (N-1)-th parts from the elapsed time. The time calculation unit 27 stores the time having required for the examination of the in the storage unit 24.

### Step S109

The time calculation unit 27 acquires the reference time table from the storage unit 24 and calculates an examination time of an endoscopic examination in a specific internal organ or a medical checkup. The examination time is the sum of an insertion time, a scheduled time for each part, and other margin time included in the reference time table. At the beginning of the examination, the time calculation unit 27 calculates a remaining time by subtracting the elapsed time from the examination time. The time calculation unit 27 calculates a standard examination time of the other parts by calculating the sum of scheduled times of parts of which an examination has not been completed. The time calculation unit 27 outputs the remaining time and the standard examination time to the time table update unit 28.

### Step S110

The region detection unit 25 detects an overlooked region in a part that has been examined before a current examination part is examined based on an image output from the storage unit 24. Accordingly, the region detection unit 25 determines whether overlooking has occurred in an examination of the examination-completed part. The region detection unit 25 outputs overlook information indicating that an overlooked region has occurred to the time table update unit 28.

For example, the region detection unit 25 executes three-dimensional (3D) reconstruction based on estimation of a camera position, image information, and the like using technology called visual simultaneous localization and mapping (visual SLAM). A 3D model is recovered through the 3D reconstruction. A position in the 3D model and a position in the image are associated.

When a partial region of the intestine does not appear in the image or when the image is not appropriate for the 3D reconstruction, a partial region of the 3D model is not recovered. The region detection unit 25 detects such a region as an overlooked region. As will be described later, the region detection unit 25 may detect an overlooked region using artificial intelligence (AI).

### Step S111

When an overlooked region has been detected by the region detection unit 25, Step S112 described later is executed. When an overlooked region has not been detected by the region detection unit 25, Step S116 described later is executed.

### Step S112

The time table update unit 28 determines whether the remaining time is within a time that is 1.1 times the standard examination time calculated in Step S109. When the remaining time is within the time that is 1.1 times the standard examination time, Step S113 described later is executed. When the remaining time is not within the time that is 1.1 times the standard examination time, Step S114 described later is executed. The numerical value of 1.1 is an example, and the present invention is not limited thereto.

### Step S113

When the remaining time is within the time that is 1.1 times the standard examination time, a part in which the overlooked region has been detected can be re-examined. The time table update unit 28 acquires the reference time table from the storage unit 24 and updates the reference time table based on the part information output from the examination position determination unit 26 and the remaining time output from the time calculation unit 27.

Details of Step S113 will be described. Based on the part information, the time table update unit 28 classifies a part of which an examination has been completed out of a plurality of parts as an examination-completed part and classifies a part of which an examination has not been completed as an examination-scheduled part. As described above, the reference time table includes the part type information indicating an examination-completed part or an examination-scheduled part. The time table update unit 28 changes the part type information of a part of which an examination has been completed. The changed part type information indicates an examination-completed part.

The time table update unit 28 re-classifies the examination-completed part in which the overlooked region has been detected as an examination-scheduled part. Accordingly, the time table update unit 28 re-classifies the examination-completed part in which it is determined that the overlooking has occurred as an examination-scheduled part. The time table update unit 28 changes the part type information of the part in which the overlooked region has been detected. The changed part type information indicates an examination-scheduled part.

It is assumed that a re-examination of the part in which an overlooked region has been detected requires the same time as a scheduled time for the part. The time table update unit 28 calculates a time (an actually remaining time) required for an examination of the other examination-scheduled parts other than that part by subtracting a time required for a re-examination of the part from the remaining time. The time table update unit 28 distributes the actually remaining time to the examination-scheduled parts according to the observation priority of each part of the large intestine. Accordingly, the time table update unit 28 updates a scheduled time of each part in the reference time table. The time table update unit 28 may distribute the actually remaining time to the parts regardless of the observation priority. In this way, the time table update unit 28 updates the reference time table in consideration of the time required for a re-examination of the part in which the overlooked region has been detected. The time table update unit 28 stores the updated reference time table (an actual examination time table) in the storage unit 24.

### Step S114

When the remaining time is not within the time that is 1.1 times the standard examination time, the examination assistance information generation unit 29 generates information for instructing the examiner to add anesthesia. This information is included in the examination assistance information generated in Step S105.

### Step S115

When anesthesia is added, a time allowed for an examination extends. Accordingly, the time table update unit 28 increases the actually remaining time. The time table update unit 28 acquires the reference time table from the storage unit 24 and distributes the actually remaining time to the parts according to the observation priority of each part of the large intestine. Accordingly, the time table update unit 28 updates the scheduled time of each part in the reference time table. The time table update unit 28 stores the updated reference time table (the actual examination time table) in the storage unit 24.

### Step S116

When the examination has been stopped or completed, the process shown in FIG. 2 ends. When the examination has not been stopped or completed, Step S103 is executed.

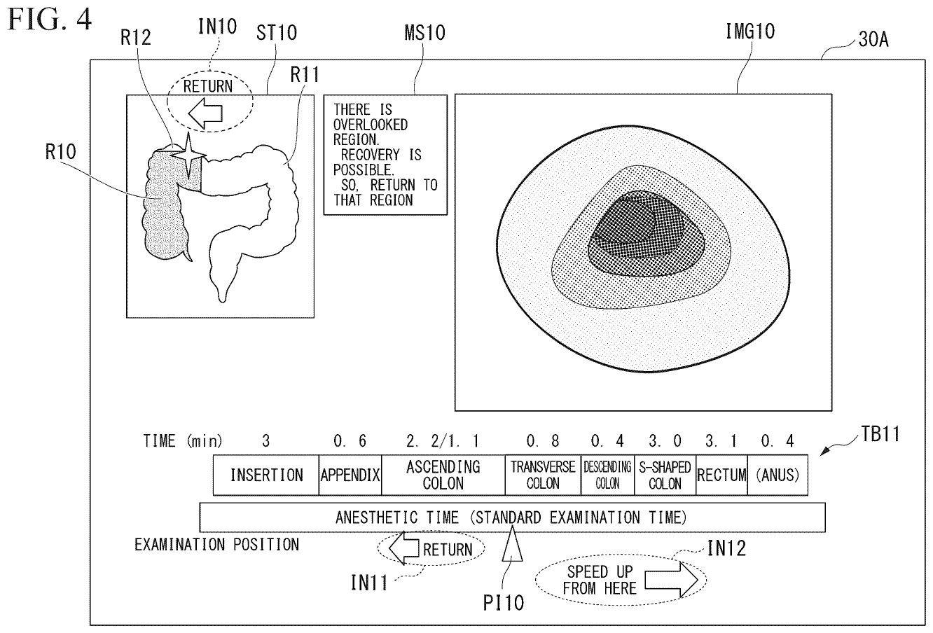

FIG. 4 shows an example of a screen of the display unit 30. The display unit 30 includes a screen 30A.

An image IMG10 output from the imaging unit 11 is displayed on the screen 30A. Examination status information ST10 indicating an examination status is displayed on the screen 30A. The examination status information ST10 is included in the examination assistance information. The examination status information ST10 indicates a region R10 including parts of which an examination has been completed, a region R11 including parts of which an examination has not been completed, and an overlooked region R12.

Instruction information IN10 and a message MS10 for encouraging an examiner to perform a re-examination are displayed on the screen 30A. The instruction information IN10 and the message MS10 are included in the examination assistance information. The instruction information IN10 intuitively encourages the examiner to return to the overlooked region and to perform a re-examination. The message MS10 uses text to encourage the examiner to return to the overlooked region and to perform a re-examination.

An actual examination time table TB11 is displayed on the screen 30A. The actual examination time table TB11 is included in the examination assistance information. The time table update unit 28 generates the actual examination time table TB11 by updating the reference time table TB10 shown in FIG. 3. The actual examination time table TB11 represents an example in which an overlooked region occurs in the ascending colon. When an examination of the ascending colon has been completed, the remaining time in the reference time table TB10 is the sum (8.8 min) of 0.9 min, 0.5 min, 3.4 min, 3.5 min, and 0.5 min.

Since an overlooked region has occurred in the ascending colon, the time table update unit 28 subtracts a time required for a re-examination of the ascending colon from the remaining time. It is assumed that the same time as the scheduled time of the ascending colon in the reference time table is required for a re-examination of the ascending colon. Since the scheduled time for the ascending colon in the reference time table is 1.1 min, the time table update unit 28 acquires an actually remaining time (7.7 min) by subtracting 1.1 min from 8.8 min. The time table update unit 28 allocates the actually remaining time (7.7 min) to the parts according to the observation priority of each part of the large intestine. Accordingly, the time table update unit 28 calculates an updated scheduled times for each part. The scheduled time for the ascending colon on which a re-examination is performed is updated with 2.2 min.

Part information PI10 indicating a current examination part is displayed on the screen 30A. The part information PI10 is included in the examination assistance information. The current examination part indicated by the part information PI10 is the transverse colon.

Instruction information IN11 and instruction information IN12 are displayed on the screen 30A. The instruction information IN11 and the instruction information IN12 are included in the examination assistance information. The instruction information IN11 is the same as the instruction information IN10. The instruction information IN12 encourages the examiner to speed up the remaining examination.

The examination assistance information generation unit 29 calculates a first sum of the scheduled times (standard examination times) in examination-scheduled parts out of the scheduled times included in the reference time table before update. For example, the examination assistance information generation unit 29 refers to the reference time table TB10 shown in FIG. 3 and calculates the first sum (8.8 min) of the scheduled times of the transverse colon to the anus. The examination assistance information generation unit 29 calculates a second sum of the scheduled times in the examination-scheduled parts out of the scheduled times included in the reference time table (the actual examination time table) after update. For example, the examination assistance information generation unit 29 refers to the actual examination time table TB11 shown in FIG. 4 and calculates the second sum (7.7 min) of the scheduled times from the transverse colon to the anus.

The examination assistance information generation unit 29 generates speed information indicating an examination speed according to a result of comparison between the first sum and the second sum. The speed information corresponds to the instruction information IN12. In the above-described example, the second sum is less than the first sum. That is, after the reference time table has been updated, the scheduled time of an examination in the remaining parts is shorter than the standard scheduled time. Accordingly, it is necessary to encourage the examiner to speed up the examination. In this case, the examination assistance information generation unit 29 generates the speed information indicating that the examination speed is to be increased. The examination assistance information generation unit 29 generates examination assistance information including the actual examination time table and the speed information and outputs the examination assistance information to the display unit 30.

The endoscopic examination assistance device 20 may execute the following process. The examination assistance information generation unit 29 refers to a part of interest (a divided section of interest) which is an examination-completed part corresponding to the current examination part in the reference time table or the actual examination time table. For example, when the current examination part indicated by the part information is an ascending colon, the part of interest is the appendix that has been examined immediately before the ascending colon is examined. The examination assistance information generation unit 29 compares a time having required for an examination of the part of interest with a scheduled time of the part of interest in the actual examination time table. The time having required for an examination of the part of interest is stored in the storage unit 24 in Step S108.

When the time having required for an examination of the part of interest is shorter than the scheduled time of the part of interest, the examination assistance information generation unit 29 generates examination assistance information including instruction information for instructing an examiner to re-examine the part of interest. For example, the examination assistance information generation unit 29 generates examination assistance information including a message for encouraging the examiner to perform a re-examination because the examination time is shorter than the scheduled time. The examination assistance information generation unit 29 outputs the examination assistance information to the display unit 30.

As described above, the reference time table includes the part type information indicating an examination-completed part or an examination-scheduled part. The time table update unit 28 changes the part type information of the part of interest. The changed part type information indicates an examination-scheduled part.

The time table generation unit 23 may determine the observation priority using statistics (overlook probabilities) of regions that the endoscope 10 is likely to overlook, or the like. Information published in a conference or the like may be used as the statistics. When the overlook probabilities are affected by differences in regional features (features such as sex, age, and race of an examinee and an examination method of an examiner who performs examination), the difference in model of the endoscope 10, or the like, the time table generation unit 23 may execute deep learning or the like according to the profile of an examinee or characteristics of an instrument or an examiner at the time of an examination and set the overlook probabilities. That is, the time table generation unit 23 may determine a part including many lesioned parts or a part in which overlooking or deterioration in visibility has occurred in a video acquired by the endoscope 10 and, for example, statistically calculate the overlook probabilities of the parts in the whole examination. The time table generation unit 23 may detect a part including many lesioned parts or the like using an inference model.

The time table generation unit 23 does not have to associate an overlooked region with a part. The time table generation unit 23 can use an inference model to infer that there is a lesioned part, that there is a frame of an image of which 3D reconstruction fails, or that there is a frame of an image of which visibility deteriorates. When illumination light is too intensive or the distal end of the endoscope 10 is hooked to an examination target, visibility deteriorates. The time table generation unit 23 may determine whether a region appearing in an image is an overlooked region based on a result of inference.

In order to generate the inference model, the time table generation unit 23 may add an annotation to a frame (a frame of interest) in which deterioration in visibility or specific image features (such as a lesioned part) have occurred out of video data including a series of frames acquired from start to end of an examination and generate training data. The time table generation unit 23 may execute learning such that a frame of interest is output when continuous frames of a video acquired by the endoscope are input and generate an inference model.

That is, the time table generation unit 23 may generate an inference model through machine learning using an image acquired by the endoscope 10 and an annotation indicating that observation of the image is not sufficient as training data. The time table generation unit 23 may determine a state of an image acquired by the endoscope 10 in an examination-scheduled part based on the inference model. The time table generation unit 23 may generate the reference time table according to the state determined based on the inference model. The time table generation unit 23 may record the generated reference time table in the storage unit 24.

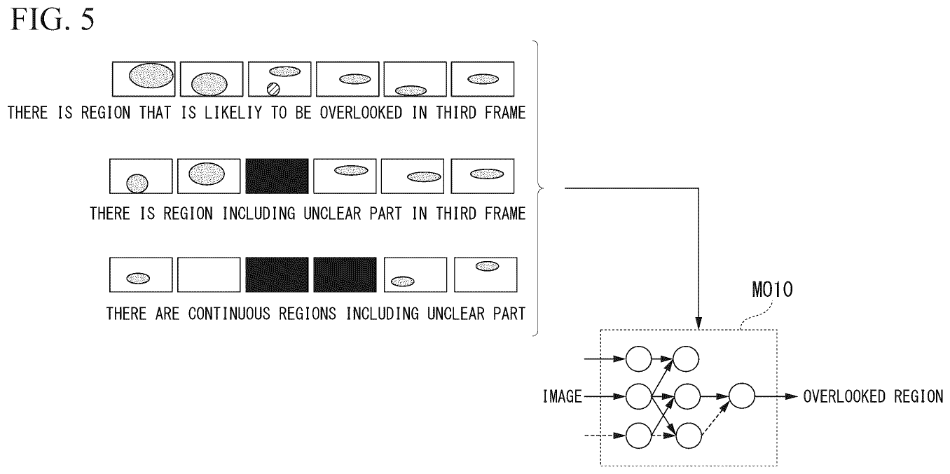

FIG. 5 shows a method of generating an inference model. The time table generation unit 23 acquires an inference model MO10 by executing machine learning in which images acquired by the endoscope 10 in a plurality of cases and annotations that are results of determination for an overlooked region in the images are used as training data. The inference model MO10 is, for example, a neural network and is generated through deep learning. The inference model MO10 is not limited to a neural network and may be another machine learning model that can output information in response to an input image. The inference model MO10 is stored in the storage unit 24.

It is assumed that the training data is acquired using a video (a series of still images) acquired when an endoscopic examination has been performed. The training data is a combination of images for training and annotations that are results of determination for an overlooked region in the images. The inference model MO10 is a model that has been trained such that an annotation corresponding to an input image is output. For example, an annotation such as “there is a region that is likely to be overlooked in a third frame” or “there is a region including an unclear part in a third frame” is used. Accordingly, it is possible to predict an overlooked region at a time point 3 frames ago. An annotation such as “there are continuous regions including an unclear part” may be used. In an example using this annotation, images to be used as the training data can be easily selected.

When abundant training data is collected, it is possible to execute good inference. The training data in the above-described example has excellent features in that specific images can be simply selected from an image group using a lesion detection technique or an image deterioration determination technique according to the related art.

In the above-described example, an annotation associated with a region that is likely to be overlooked or an unclear region is used. Since a lesioned part appears in an image subsequent to an image having specific features, an annotation for attracting an examiner's attention may be used for avoiding overlooking of the image. Alternatively, an annotation for attracting an examiner's attention may be used for avoiding overlooking of an image less likely to be used for 3D reconstruction.

The time table generation unit 23 may input a plurality of images output from the imaging unit 11 to the inference model MO10 and identify an image determined to include an region as an overlooked region. The time table generation unit 23 may calculate the overlook probability of each part based on the identified image in the part. The time table generation unit 23 may set observation priorities according to the overlook probabilities of the parts and generate the reference time table according to the observation priorities.

In a case where an image acquired by the endoscope 10 is used as training data while an examiner is performing an operation of inserting the endoscope 10 into an intestine, the time table generation unit 23 executes inference while the operation is being performed and generates the reference time table. On the other hand, the time table generation unit 23 may execute inference while the examiner is extracting the endoscope 10 from the intestine. In this case, an annotation is added to an image acquired immediately after extraction of the endoscope 10 has started in order to execute inference of a prediction system, and then training is executed. After the extraction has started and a specific period of time has elapsed, the time table generation unit 23 can generate the reference time table.

“Deep learning” contains processes of “machine learning” using a neural network, and the processes are structured in multiple layers. A representative example is a “forward-propagation neural network” of executing determination while sending information forwardly. In the most simple example, the neural network has only to include three layers that are an input layer including N1 neurons, an intermediate layer including N2 neurons given as parameters, and an output layer including N3 neurons corresponding to the number of classes to be determined. By coupling the neurons in the input layer and the intermediate layer by coupling weights, coupling the neurons in the intermediate layer and the output layer by coupling weights, and adding bias values to the intermediate layer and the output layer, logic gates can be easily formed. Three layers may be used for the purpose of simple determination, and a method of combining a plurality of features may be learned in the course of machine learning by increasing the number of intermediate layers. In recent years, 9 to 152 intermediate layers can be practically used in view of a time required for learning, determination accuracy, and energy consumption. A “convolutional neural network” using minimum processes accompanying a process called “convolution” of compressing features of an image may be used. The convolutional neural network is strong in recognition of motion and patterns. Alternatively, a “recurrent neural network” (an all-coupling recurrent neural network) that can handle more complex information may be used. In the recurrent neural network, information propagates bidirectionally to correspond to information analysis in which meanings change according to the sequence. In addition, a technique such as support vector machine or support vector recurrence is used as a pattern recognition model using supervised learning. Weights, filter coefficients, and offsets of discriminators are calculated in such learning. In addition, there is also a technique using a logistic regression process.

A general-purpose arithmetic processing circuit such as a CPU or a field-programmable gate array (FPGA) may be used to execute learning, and a circuit such as a graphic processing unit (GPU) or a tensor processing unit (TPU) characterized in matrix calculation may be used since most processes in the neural network are multiplication of matrices. In recent years, a “neural network processing unit (NPU)” that is hardware specific to artificial intelligence (AI) may be designed to be integrated with a circuit such as a CPU and serve as a part of a processing circuit.

For example, when an inference model generated through learning using only images acquired by general imaging and having the horizontal and vertical directions aligned is used, there is a likelihood that correct inference may not be performed on images having vertical or horizontal differences. Accordingly, it is possible to perform correct determination by reading the above-described information from inference information and executing inference on images acquired in consideration of horizontal or vertical information from a posture sensor. An idea of adding horizontal and vertical information and determining images before executing inference using the inference model is effective. It is preferable that the endoscope system 1 store information of such conditions and have a sensor for correcting an image. Specifications and performance of an inference engine change according to whether such constraints are added at the time of learning. Accordingly, this trial and error may be performed in parallel with an annotation operation, or the trial and error may be displayed.

Similarly, when learning using only images captured at a position separated by a specific distance from an object is executed, correct inference cannot be executed on an image captured at a position separated by a distance other than the specific distance. In inference using an inference model generated based on such images, it is possible to improve accuracy based on an idea of enlarging an image of a far object and artificially using an image of a nearby object in order to cancel the difference in distance. In this case, a distance sensor or the like is used together, and correction for complementing the difference between a state of actual enlargement or reduction of an image and a state of training data is executed at the time of inference of the image. The endoscopic examination assistance device 20 may include a memory for storing information indicating the training data used to generate the inference model and correct an image such that the inference model can correctly execute inference using the above-described information when the inference using the inference model is executed. A user may be aware whether such correction is necessary in the annotation operation. As in the present embodiment, the idea of enabling verification of provisional learning in the annotation operation is significant.

It is difficult for a compact inference engine mounted in an information terminal product such as a camera or a portable device to execute learning for highly accurate determination with smaller layers. Since learning requires a time, there is need for an idea associated with a method of executing accurate annotation and learning. When an inference model is generated, specifications of the inference model are changed according to images used for learning, and thus efficient learning may be executed in cooperation with information at the time of learning. Therefore, information indicating what learning has been executed may be set in the annotation operation, and this information may be recorded as part of inference information in a recording unit of an information acquisition device.

As described above, in “supervised learning,” a “relationship between an input and an output” is learned using training data of which the output is determined by an annotation, and inference with high reliability under specific conditions is executed. On the other hand, the endoscopic examination assistance device 20 may acquire an inference model that can cope with more complex situations using a technique of “unsupervised learning” of learning a “data structure.”

The endoscopic examination assistance device 20 may use a technique of learning an “action for maximizing values and effects” called “reinforcement learning.” In this technique, learning is executed such that a rule for enhancing state/action values is searched for. Trial and error are made until values of a next state other than a current state are estimated and enhanced or specific rewards are acquired, and the results of the trial and error are reflected in learning. Training data may be used to verify results of learning. In this technique, the output of an answer acquired by the annotation is not learned as it is, but learning is executed such that a more correct answer can be obtained. Accordingly, it is possible to cope with an unknown situation.

This inference may be used together with supervised learning, or the inference may be executed using supervised learning after the inference has been executed using unsupervised learning. Annotation data can also be used as verification data for such “unsupervised learning” and “reinforcement learning.”

When a machine is made to determine something, a human being needs to teach the machine a determination method. Here, a technique of executing determination of an image through machine learning has been employed, and a rule-based technique of causing a human being to apply an experimental rule or a rule acquired in heuristics to determination may be used.

As described above, the endoscopic examination assistance device 20 can output an examination time table (an actual examination time table) associated with times required for examination-scheduled sections (parts) to recover examination.

### Second Embodiment

A second embodiment of the present invention will be described. In the second embodiment, the endoscope system 1 shown in FIG. 1 is used.

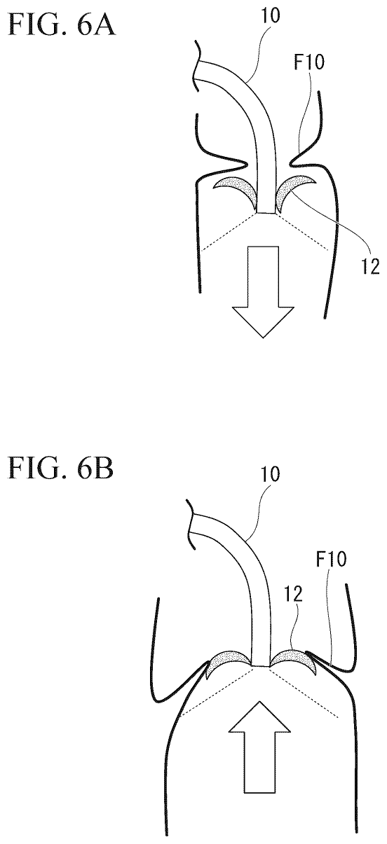

An adapter (an end cuff) for turning over a fold in a large intestine is attached to the distal end of the endoscope 10. FIGS. 6A and 6B show a state of the endoscope 10 to which an adapter 12 is attached. FIG. 6A shows a state in which an examiner inserts the endoscope 10 into a large intestine and the endoscope 10 is advancing in a depth direction in the large intestine. FIG. 6B shows a state in which the examiner is performing examination while extracting the endoscope 10. In a part in which a fold F10 is present, the distal end of the endoscope 10 moves while the adapter 12 slowly turns over the fold F10, which requires time. Accordingly, the endoscopic examination assistance device 20 changes the reference time table generated based on the assumption that the adapter 12 is not used.

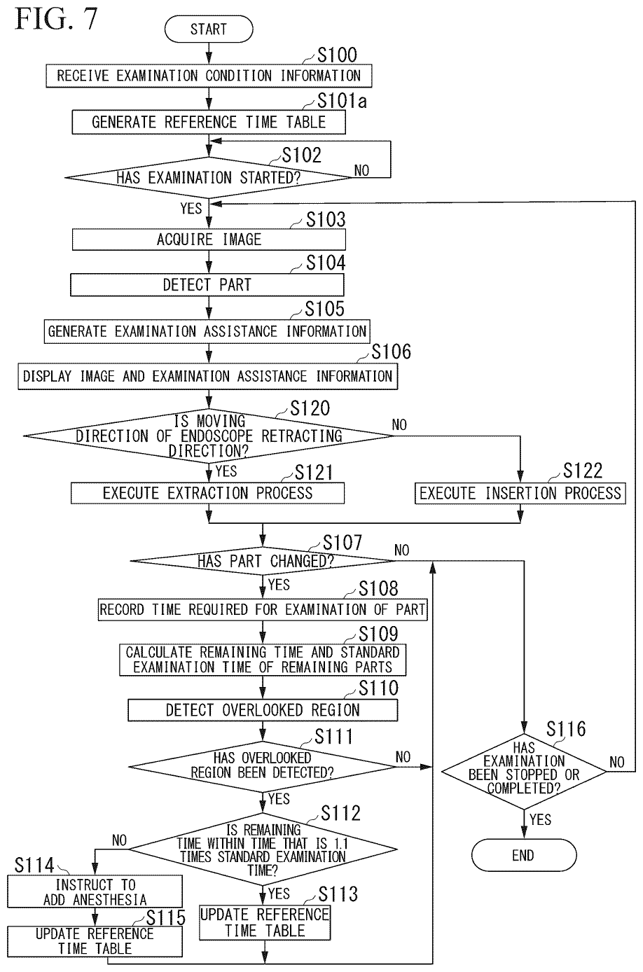

An example of operations of the endoscopic examination assistance device 20 will be described with reference to FIG. 7. FIG. 7 shows an example of a procedure of a process executed by the endoscopic examination assistance device 20. The same processes as those shown in FIG. 2 will not be described. Processes different from those shown in FIG. 2 will be described.

### Step S101a

Step S101 shown in FIG. 2 is changed as follows. When the adapter 12 is used, the time table generation unit 23 generates a reference time table by executing the same process as Step S101 shown in FIG. 2. Thereafter, the time table generation unit 23 updates the reference time table. When the adapter 12 is used, a time required for insertion of the endoscope 10 and a time required for an examination of a specific part increase. Accordingly, the time table generation unit 23 increases such times in the reference time table and decreases times required for an examination of other parts. The sum of times that are increased and the sum of times that are decreased are the same. The time table generation unit 23 stores the updated reference time table in the storage unit 24.

The endoscopic examination assistance device 20 may detect features of an image change (image features) at the time of insertion of the endoscope 10 and change a weight of importance of an examination at the time of extraction of the endoscope 10 based on the image features. When an examination plan in which the endoscope 10 is inserted into a lumen of an internal organ and an examination is performed at the time of extraction of the endoscope 10 is used, the endoscopic examination assistance device 20 can employ an idea of detecting the change of the image features in each of a plurality of divided sections, storing information of the detected image features in the storage unit 24, and switching a time distribution of an examination to each divided section of the internal organ using the information of the image features stored in the storage unit 24. For example, the information of the image features indicates whether there is a lesioned part or whether visibility is good. This is an idea indicating that a section of which an examination is important can also be determined to some extents at the time of insertion of the endoscope 10.

### Step S120

After Step S106 has been executed, the examination position determination unit 26 determines a moving direction of the endoscope 10 based on a plurality of images output from the storage unit 24. When an examiner is moving the endoscope 10 toward an examination target in the intestine, the moving direction of the endoscope 10 is the depth direction. When the examiner is moving the endoscope 10 in a direction in which the endoscope is extracted from the intestine, the moving direction of the endoscope 10 is a retracting direction. When the moving direction of the endoscope 10 is the retracting direction, Step S121 is executed. When the moving direction of the endoscope 10 is the depth direction, Step S122 is executed.

### Step S121

The endoscopic examination assistance device 20 executes an extraction process. Details of the process will be described later. After Step S121 has been executed, Step S107 is executed.

### Step S122

The endoscopic examination assistance device 20 executes an insertion process. For example, the region detection unit 25 determines whether a region appearing in an image output from the storage unit 24 is a fold region. The result of determination is stored in the storage unit 24. The region detection unit 25 detects a lesioned part such as a polyp appearing in the image output from the storage unit 24. Information of the detected lesioned part is associated with the part information generated in Step S104 and is stored in the storage unit 24. Accordingly, information of a lesioned part in each part is stored in the storage unit 24. After Step S122 has been executed, Step S107 is executed.

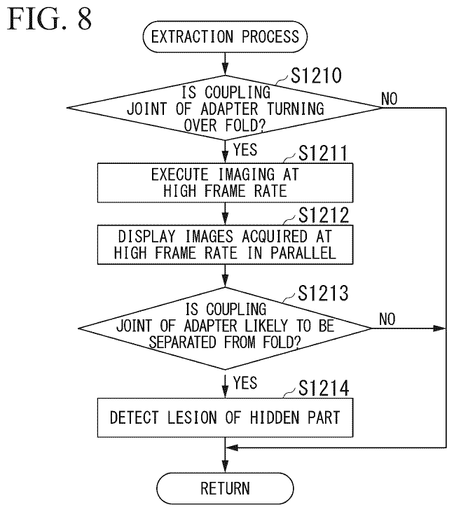

FIG. 8 shows an example of a procedure of a process executed by the endoscopic examination assistance device 20 in Step S121. An example of the operation of the endoscopic examination assistance device 20 in Step S121 will be described below.

### Step S1210

The region detection unit 25 determines whether a coupling joint of the adapter 12 is turning over a fold based on the image output from the storage unit 24. When the coupling joint is turning over a fold, Step S1211 described later is executed. When the coupling joint is not turning over a fold, Step S107 shown in FIG. 7 is executed.

### Step S1211

When the coupling joint is turning over a fold, the imaging unit 11 executes imaging at a high frame rate. The imaging unit 11 outputs two or more images. The image acquisition unit 21 acquires the two or more images output from the imaging unit 11 and outputs the acquired two or more images to the storage unit 24. The storage unit 24 stores the two or more images.

### Step S1212

The storage unit 24 outputs the two or more images generated in Step S1211 to the display unit 30. The display unit 30 displays the two or more images in parallel.

### Step S1213

The region detection unit 25 determines whether the coupling joint of the adapter 12 is likely to be separated from the fold based on the image output from the storage unit 24. When the coupling join is likely to be separated from the fold, Step S1214 described later is executed. When the coupling joint is not likely to be separated from the fold, Step S107 shown in FIG. 7 is executed.

### Step S1214

The region detection unit 25 detects a lesioned part of a region newly appearing in the image as the coupling joint of the adapter 12 is gradually separated from the fold based on the image output from the storage unit 24.

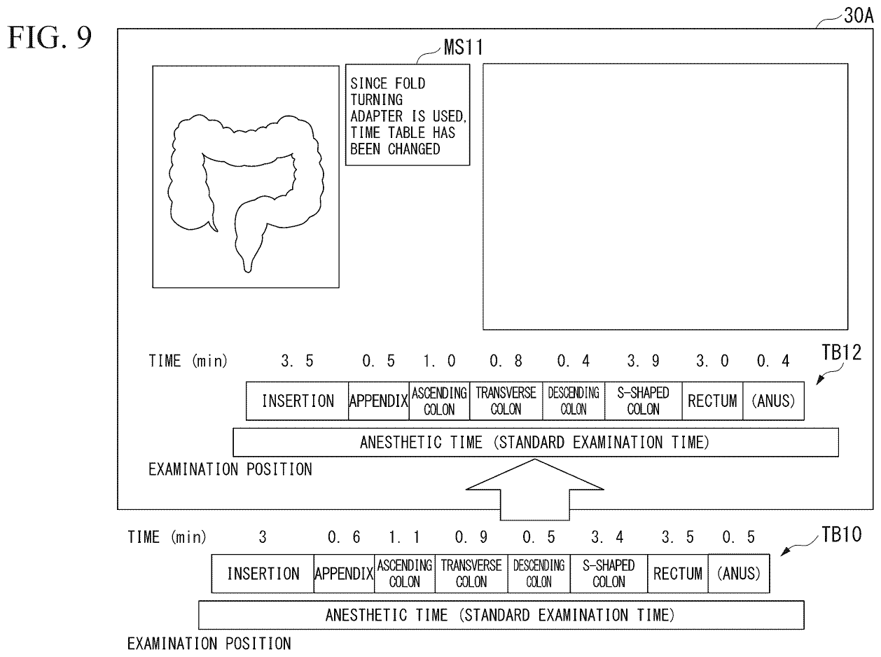

FIG. 9 shows an example of the screen 30A of the display unit 30. When the adapter 12 is used, the time table generation unit 23 changes the reference time table in Step S101a shown in FIG. 7. The changed reference time table TB12 is displayed on the screen 30A. The reference time table TB12 is included in the examination assistance information.

The time table generation unit 23 generates the reference time table TB12 from the reference time table TB10 shown in FIG. 3. For example, the time table generation unit 23 increases the insertion time (3 min) to 3.5 min and increases the scheduled time (3.4 min) required for an examination of the S-shaped colon to 3.9 min. Since the sum of the insertion time and the scheduled time for the S-shaped colon is increased by 1 min, the time table generation unit 23 decrease the sum of the scheduled times of the parts other than the S-shaped colon by 1 min.

A message MS11 is displayed on the screen 30A. The message MS11 is included in the examination assistance information. The message MS11 indicates that the reference time table TB10 has been changed due to use of the adapter 12.

When a region appearing in the image acquired at the time of extraction of the endoscope 10 is the same as a region appearing in the image acquired at the time of insertion of the endoscope 10, the endoscope 10 may pass through the region fast. When a part in which an overlooked region has occurred is re-examined, it is possible to recover the examination by causing the examiner to move the endoscope 10 fast in a region without any change.

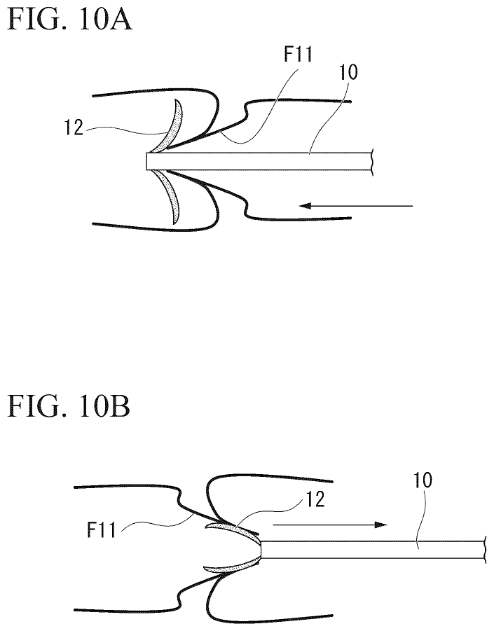

FIGS. 10A and 10B show states of the endoscope 10 to which the adapter 12 is attached. FIG. 10A shows a state in which an examiner inserts the distal end of the endoscope 10 into a large intestine and the distal end of the endoscope 10 is moving in the depth direction in the large intestine. FIG. 10B shows a state in which the examiner is performing an examination while extracting the endoscope 10. In a part in which a fold F11 is present, a part of the fold F11 appearing in the image shown in FIG. 10A is different from that of the fold F11 appearing in the image shown in FIG. 10B. On the other hand, parts other than the folder F11 appearing in the image shown in FIG. 10A are the same as those other than the fold F11 appearing in the image shown in FIG. 10B. Since the parts other than the fold F11 are observed at the time of insertion of the endoscope 10, the examiner can move the distal end of the endoscope 10 fast in the parts.

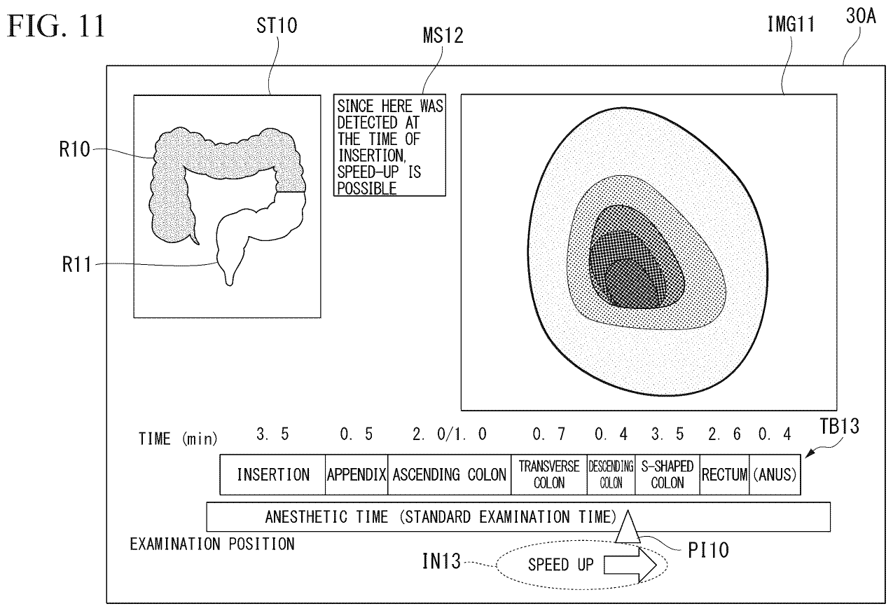

FIG. 11 shows an example of the screen 30A of the display unit 30. The same parts as those shown in FIG. 4 will not be described. Parts different from those shown in FIG. 4 will be described.

An image IMG11 output from the imaging unit 11 is displayed on the screen 30A. An actual examination time table TB13 is displayed on the screen 30A. The actual examination time table TB13 is included in the examination assistance information. The time table update unit 28 generates the actual examination time table TB13 by updating the reference time table TB12 shown in FIG. 9. The actual examination time table TB13 indicates an example in which an overlooked region occurs in the ascending colon.

Since an overlooked region has occurred in the ascending colon, the scheduled time of the ascending colon in the actual examination time table TB13 is changed from 1.0 min to 2.0 min. Since the scheduled time of the ascending colon is increased by 1 min, the time table update unit 28 decreases the sum of the scheduled times of other parts of which an examination has not been completed by 1 min.

The same part information PI10 as the part information PI10 shown in FIG. 4 is displayed on the screen 30A. The current examination part indicated by the part information PI10 is a descending colon. Since a part of the descending colon appearing in the image at the time of extraction of the endoscope 10 is the same as that of the descending colon appearing in the image at the time of insertion of the endoscope 10, the examiner may move the distal end of the endoscope 10 fast in the part. Accordingly, instruction information IN13 and a message MS12 are displayed on the screen 30A. The instruction information IN13 and the message MS12 are included in the examination assistance information. The instruction information IN13 intuitively encourages the examiner to move the endoscope 10 fast. The message MS12 encourages the examiner to move the endoscope 10 fast by text.

Information of a lesioned part detected in Step S122 at the time of insertion of the endoscope 10 is stored in the storage unit 24. At the time of extraction of the endoscope 10, the examiner performs treatment of the lesioned part. When treatment of the lesioned part detected in Step S122 has not been executed, the region detection unit 25 detects the lesioned part as an overlooked region.

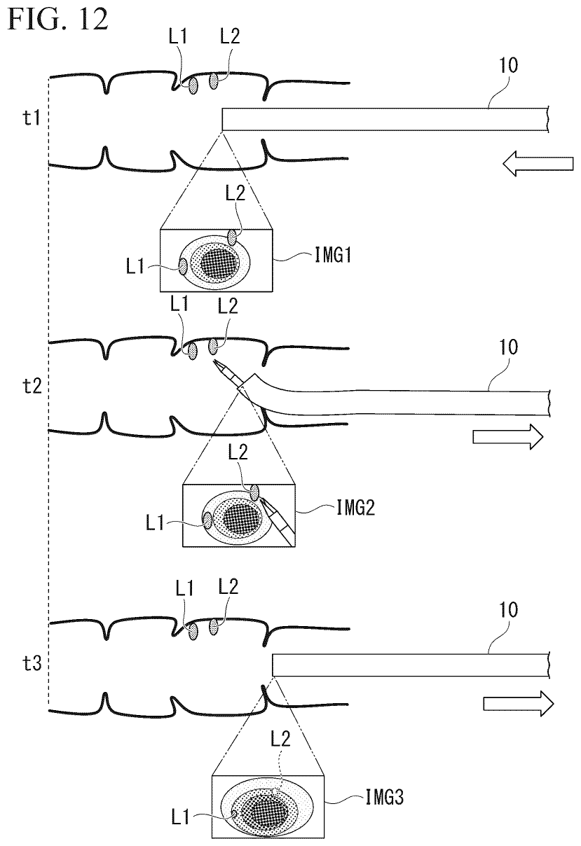

FIG. 12 shows an example in which a lesioned part is treated. The imaging unit 11 generates an image IMG1 at an imaging time t1, generates an image IMG2 at an imaging time t2, and generates an image IMG3 at an imaging time t3. When the endoscope 10 is moving in the depth direction, the imaging unit 11 generates the image IMG1. When the endoscope 10 is moving in the retracting direction, the imaging unit 11 generates the image IMG2 and the image IMG3.

When the endoscope 10 is moving in the depth direction, the region detection unit 25 detects a lesioned part L1 and a lesioned part L2 appearing in the image IMG1. Information of the lesioned part L1 and the lesioned part L2 is stored in the storage unit 24.

When the endoscope 10 is moving in the retracting direction, the region detection unit 25 detects the lesioned part L1 and the lesioned part L2 appearing in the image IMG2. The examiner performs treatment on the lesioned part L2 and forgets that treatment is to be performed on the lesioned part L1. Information indicating that treatment has been performed on the lesioned part L2 is detected from an image generated by the imaging unit 11. Information indicating that treatment has been performed on the lesioned part L2 and information indicating that treatment has not been performed on the lesioned part L1 are stored in the storage unit 24. The region detection unit 25 detects the lesioned part L1 appearing in the image IMG3. The lesioned part L2 is hidden by a fold and does not appear in the image IMG3.

The region detection unit 25 acquires information indicating whether treatment has been performed on a lesioned part from the storage unit 24 in Step S110 and detects an overlooked region based on the acquired information. In the above-described example, the region detection unit 25 determines that the lesioned part L2 is not an overlooked region based on the information indicating that treatment has been performed on the lesioned part L2. The region detection unit 25 determines that the lesioned part L1 is an overlooked region based on the information indicating that treatment has not been performed on the lesioned part L1.

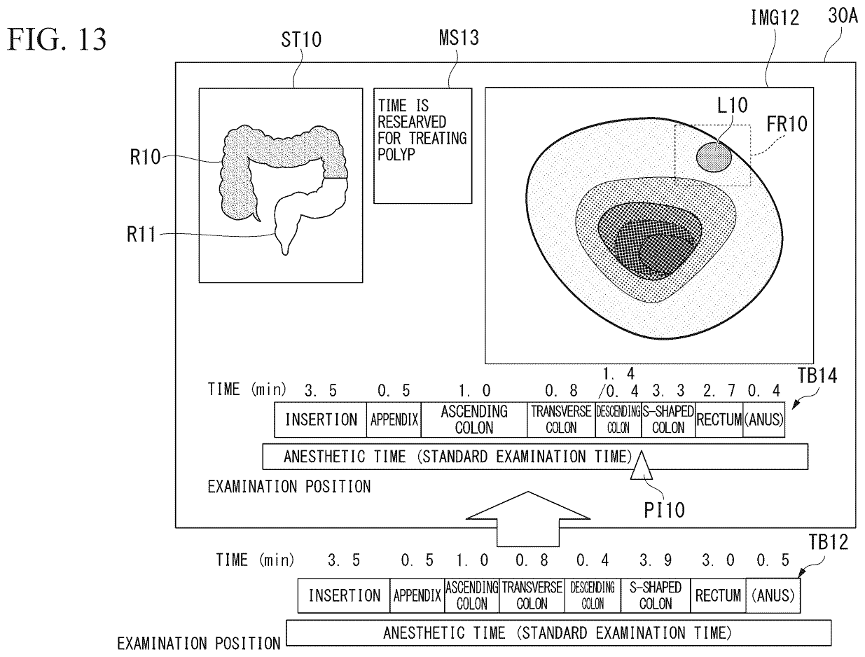

FIG. 13 shows an example of the screen 30A of the display unit 30. The same parts as those shown in FIG. 4 will not be described. Parts different from those shown in FIG. 4 will be described.

An image IMG12 output from the imaging unit 11 is displayed on the screen 30A. A frame FR10 indicating a position of a lesioned part L10 detected by the region detection unit 25 is superimposed on the image IMG12.

When the endoscope 10 is extracted from a lumen of an internal organ, the region detection unit 25 detects a lesioned part on which treatment has not been performed as an overlooked region. The time table update unit 28 determines whether a lesioned part has been overlooked in an examination of an examination-completed part using information stored in the storage unit 24. The time table update unit 28 re-classifies the examination-completed part including the lesioned part determined to have been overlooked as an examination-scheduled part. As described above, the reference time table includes the part type information indicating an examination-completed part or an examination-scheduled part. The time table update unit 28 changes the part type information of the part including the lesioned part determined to have been overlooked. The changed part type information indicates the examination-scheduled part. When the examination assistance information generation unit 29 outputs an updated reference time table to the display unit 30, the display unit 30 displays a remaining time and a time distribution of an examination in the examination-scheduled parts.

The time table update unit 28 generates an actual examination time table by updating the reference time table in Step S113 shown in FIG. 7. An actual examination time table TB14 is displayed on the screen 30A. The actual examination time table TB14 is included in the examination assistance information.

The time table update unit 28 generates the actual examination time table TB14 from the reference time table TB12 shown in FIG. 9. The actual examination time table TB14 indicates an example in which an overlooked region occurs in the descending colon.

Since an overlooked region has occurred in the descending colon, the scheduled time of the descending colon in the actual examination time table TB14 is changed from 0.4 min to 1.4 min. Since the scheduled time of the descending colon is increased by 1 min, the time table update unit 28 decreases the sum of the scheduled times of other parts of which an examination has not been completed by 1 min.

A message MS13 is displayed on the screen 30A. The message MS13 is included in the examination assistance information. The message MS13 encourages the examiner to perform treatment on the lesioned part by text.

As described above, when the adapter 12 is attached, the reference time table is updated. Even when treatment on a lesioned part has not been performed and the lesioned part has been detected as an overlooked region, the reference time table is updated. Accordingly, the endoscopic examination assistance device 20 can output an examination time table associated with times required for examination-scheduled sections (parts) in order to recover the examination.

When a re-examination of a part in which an overlooked region has occurred is performed, the display unit 30 displays information for encouraging the examiner to move the distal end of the endoscope 10 fast. The examiner can move the distal end of the endoscope 10 fast in a region without any change, and thus it is possible to recover the examination.

While preferred embodiments of the invention have been described and shown above, it should be understood that these are examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An endoscopic examination assistance method comprising:
calculating a remaining time until an examination of an internal organ using an endoscope ends based on a preset examination time for the examination and an elapsed time having elapsed after the examination has started;
acquiring current position information indicating a current examination position in the internal organ; and
generating and outputting an examination time table including a scheduled examination time for each of a plurality of sections in the internal organ using the remaining time and the current position information.

2. The endoscopic examination assistance method according to claim 1, further comprising:
classifying each of the plurality of sections as an examination-completed section of which the examination has been completed or an examination-scheduled section of which the examination has not been completed using the current position information; and
generating and outputting the examination time table including the scheduled examination time for each examination-scheduled section according to the remaining time.

3. The endoscopic examination assistance method according to claim 2, further comprising:
determining whether overlooking has occurred in an examination of the examination-completed section; and
re-classifying the examination-completed section in which it is determined that the overlooking has occurred as the examination-scheduled section at the time of generation and output of the examination time table.

4. The endoscopic examination assistance method according to claim 2, further comprising:
detecting a lesioned part in each of the plurality of sections when the endoscope is inserted into a lumen of the internal organ;
storing information of the detected lesioned part in a storage medium;
determining whether overlooking of the lesioned part has occurred in an examination of the examination-completed section using the information stored in the storage medium when the endoscope is pulled out from the lumen; and
re-classifying the examination-completed section including the lesioned part in which it is determined that the overlooking has occurred as the examination-scheduled section at the time of generation and output of the examination time table.

5. The endoscopic examination assistance method according to claim 2, further comprising:
referring to a section of interest which is the examination-completed section corresponding to the current position information in the examination time table; and
outputting instruction information for instructing an examiner to re-examine the section of interest when a time having required for an examination in the section of interest is shorter than the scheduled examination time of the section of interest.

6. The endoscopic examination assistance method according to claim 5, further comprising:
re-classifying the section of interest as the examination-scheduled section; and
updating and outputting the examination time table based on a time required for the re-examination.

7. The endoscopic examination assistance method according to claim 2, further comprising:
acquiring a reference time table including the scheduled examination time for each of the plurality of sections from a storage medium;
executing a process of updating the scheduled examination time in the examination-scheduled section on the reference time table using the remaining time and the current position information; and
outputting the updated reference time table as the examination time table.

8. The endoscopic examination assistance method according to claim 7, further comprising:
determining states of images acquired by the endoscope in the plurality of sections based on an inference model acquired through machine learning using images acquired by the endoscope and annotations indicating that observation of the images is insufficient as training data;
generating the reference time table according to the states determined based on the inference model; and
recording the generated reference time table in the storage medium.

9. The endoscopic examination assistance method according to claim 7, further comprising:
calculating a first sum of the scheduled examination times in the examination-scheduled sections out of the scheduled examination times included in the reference time table before update;
calculating a second sum of the scheduled examination times in the examination-scheduled sections out of the scheduled examination times included in the reference time table after update;
generating speed information indicating an examination speed according to a result of comparison between the first sum and the second sum; and
outputting the examination time table and the speed information to a display.

10. The endoscopic examination assistance method according to claim 1, further comprising:
calculating the scheduled examination time for each examination-scheduled section according to an adapter attached to a distal end of the endoscope; and
generating and outputting the examination time table including the calculated scheduled examination time.

11. The endoscopic examination assistance method according to claim 1, further comprising outputting the examination time table and the current position information to a display.

12. An endoscopic examination assistance device comprising a processor configured to:
calculate a remaining time until an examination of an internal organ using an endoscope ends based on a preset examination time for the examination and an elapsed time having elapsed after the examination has started;
acquire current position information indicating a current examination position in the internal organ; and
generate and output an examination time table including a scheduled examination time for each of a plurality of sections in the internal organ using the remaining time and the current position information.

13. A non-transitory computer-readable recording medium storing a program causing a computer to execute:
calculating a remaining time until an examination of an internal organ using an endoscope ends based on a preset examination time for the examination and an elapsed time having elapsed after the examination has started;
acquiring current position information indicating a current examination position in the internal organ; and
generating and outputting an examination time table including a scheduled examination time for each of a plurality of sections in the internal organ using the remaining time and the current position information.

14. An endoscopic examination assistance method comprising:
calculating a remaining time until an examination of an internal organ using an endoscope ends based on a preset examination time for the examination and an elapsed time having elapsed after the examination has started;
acquiring current position information indicating a current examination position in the internal organ; and
generating and outputting an examination time table including a scheduled examination time for each of a plurality of sections in the internal organ for simultaneously displaying the remaining time and the current position information.

15. The endoscopic examination assistance method according to claim 14, further comprising displaying the remaining time and a time distribution for an examination in each part of the internal organ in at least a region in which the remaining time is displayed based on the examination time table.

16. The endoscopic examination assistance method according to claim 15, wherein the time distribution is determined according to a difference between surface areas of examination target parts of the internal organ to be examined, statistics of lesion occurrence of the examination target parts, or examination difficulty based on a shape of each examination target part.

17. The endoscopic examination assistance method according to claim 14, further comprising resetting the time distribution for the examination in each part of the internal organ based on the remaining time when an examination delay exceeding a predetermined time width is detected in a time allocated to the examination of each part of the internal organ.

18. The endoscopic examination assistance method according to claim 15, further comprising:
classifying each of the plurality of sections as an examination-completed section of which the examination has been completed or an examination-scheduled section of which the examination has not been completed using the current position information;
detecting a lesioned part in each of the plurality of sections when the endoscope is inserted into the internal organ;
storing information of the detected lesioned part in a storage medium;
determining whether overlooking of the lesioned part has occurred in an examination of the examination-completed section using the information stored in the storage medium when the endoscope is extracted from the internal organ;
re-classifying the examination-completed section including the lesioned part in which it is determined that the overlooking has occurred as the examination-scheduled section at the time of generation and output of the examination time table; and
displaying the remaining time and the time distribution for the examination in the examination-scheduled section.

19. The endoscopic examination assistance method according to claim 15, further comprising:
classifying each of the plurality of sections as an examination-completed section of which the examination has been completed or an examination-scheduled section of which the examination has not been completed using the current position information;
detecting an image feature in each of the plurality of sections when the endoscope is inserted into a lumen of the internal organ;
storing information of the detected image feature in a storage medium; and
switching the time distribution for the examination in each part of the internal organ using the information of the image feature for each section stored in the storage medium.

20. The endoscopic examination assistance method according to claim 15, further comprising:
referring to the examination time table; and
switching the time distribution for the examination in each part of the internal organ when a time having required for the examination of a specific section out of the plurality of sections in the internal organ is short.
